(19) United States
(12) Patent Application Publication
(10) Pub. No.: US 2018/0199857 A1
(43) Pub. Date: Jul. 19, 2018

(54) ANTISNORING DEVICE

(71) Applicant: Hamid BAYAN, Duesseldorf (DE)

(72) Inventor: Hamid BAYAN, Duesseldorf (DE)

(21) Appl. No.: 15/554,848

(22) PCT Filed: Jan. 30, 2014
(86) PCT No.: PCT/EP2014/051844

§ 371 Date: Mar. 31, 2018

(30) Foreign Application Priority Data
Feb. 25, 2013 (DE) 10 2013 203 081.9
Apr. 26, 2013 (DE) 10 2013 207 635.5

(51) Int. Cl. A61B 5/11; A61B 5/00

(52) U.S. Cl. CPC A61B 5/1116; A61B 5/4806; G08B 21/06; A61B 5/6805; A61B 5/6823

(57) ABSTRACT

The invention relates to a device (1, 1′, 1″) for detecting an unhealthy situation of a person lying down, wherein means for sensing the position of the person lying down are provided and there are further means that cause the person to react when an unhealthy situation of the person lying down has been sensed by the means for sensing the position, wherein the means for sensing the position of the person lying down are configured as at least one switching means (8, 9) and wherein the means that cause the person to react are configured as a vibrating means (3) and wherein a housing (7) accommodates at least the vibrating means (3), a power supply (4) and an input/output means (5) and also the at least one switching means (8, 9) and the housing (7) are fixed to a band (6).

The invention relates to an apparatus for detecting an unhealthy position of a recumbent person, in particular an antisnoring apparatus according to the features of the preamble of the independent claims.

For a wide range of reasons, unhealthy positions can occur in people who are recumbent, particularly in bed. In this case, an “unhealthy position” is understood to refer to one in which a recumbent person, and due to an illness (for example arthritis in the shoulder region), is able to lie (particularly sleep) without any problems on his/her back, face-down, or on one side, whereas lying on the other side impedes sleep or even promotes illness, that is, it is unhealthy overall.

Moreover, an “unhealthy” position is to be understood as a position in which a person is recumbent and breathing arrest (apnea), snoring, or the like can occur. In the worst-case scenario, such an unhealthy position would be a person with osteoarthritis in the shoulder region and a tendency to experience apnea when lying on his back.

US 2009/0250070 and DE 10 2008 016 855 disclose an apparatus and a method of influencing a person's sleep. The apparatus comprises a sensor for detecting a current rotational position of the body of the person about the longitudinal axis of the body, an actuator for supplying a predetermined stimulus pattern to the body, and an operating unit with a selection apparatus for selecting at least one desired and/or at least one prohibited rotational position of the body of the person about the longitudinal axis of the body, a comparator for comparing the current rotational position and the at least one desired and/or at least one prohibited rotational position and emitting a corresponding position signal, and a controller that can be set to actuate the actuator to supply the predetermined stimulus pattern to the body in response to the position signal if the position signal shows that the current rotational position does not correspond to the at least one desired rotational position and/or corresponds to at least the one prohibited rotational position. This apparatus has a very elaborate construction.

US 2010/0051037 discloses functional clothing for the prevention of sleep-related breathing disorders and/or decubitus that is worn by a user. The clothing has a plurality of air chambers on the back that can be filled and emptied. An air supply feeds air to each of the air chambers, and a pressure detector measures the pressure in each of the air chambers. An electrical signal is generated from the measured pressure. A control system receives the measured pressure signal from the pressure detector. Using the air supply unit, the air chambers are filled when snoring or sleep apnea is detected in the user or a predetermined amount of time has passed in which the user has remained in the same position in order to change the user's position. The air chambers can be damaged and thus lose his/her functionality.

US 2011/0132378 discloses systems and methods for controlling the position of a user of a wearable positional therapy apparatus. The wearable position therapy apparatus is configured to monitor and store physiological signals that can be used to assess the sleep quality and sleeping position of a user. The apparatus can be configured to be worn around the head, the neck, or body of the user. Moreover, the apparatus can provide feedback to a user if the user is sleeping or is positioned in a target position to induce the user to change positions. The feedback can be provided by one or more haptic motors that can be configured to provide various levels of feedback and the level of feedback can be customized based on the user's reaction to the feedback. Due to the use of a microprocessor, this apparatus has a very elaborate construction.

WO 2011/139141 [U.S. Pat. No. 9,060,880] discloses a sleep posture alerting method and apparatus. The sleep posture of a person is detected and the person is automatically alerted when the sleep posture is out of a predetermined body posture range. The detection of the sleep posture or at least the alerting of the person when his/her body posture is out of the predefined body posture range, is deactivated during a sleep-in time period. This apparatus is also based on control by a microprocessor and is therefore highly elaborate and error-prone.

DE 101 20 922 relates to a method of eliminating snoring in which an acoustic detector detects the snoring noise and, upon detection, a vibrator worn by the snorer on his body is caused to vibrate. This apparatus is based on an acoustic detector for snoring noises and is therefore very sensitive to snoring noises.

DE 198 09 470 relates to the use of a sensor and signaling means for monitoring the position in which a person is lying, this detection being based on the fact that substantial snoring noises occur in the person in question above all when sleeping in a position other than on the side and that such snoring noise may be associated with breathing arrests that can result in death and substantially increase the risk of sudden infant death syndrome in babies and small children. Through the use of the sensor for detecting the position and/or movement of a recumbent person on the one hand and signal means on the other hand that work together with the sensor to directly and/or indirectly influence the position of this person when positional changes are observed, the person himself is made to assume the side position that inhibits snoring, or a small child whose position is being monitored in order to reduce the risk of sudden infant death syndrome can be moved by a care giver who receives the signal from the risky back position to the safe side position. This apparatus is based on inclination sensors for monitoring the recumbent position, which have elaborate constructions and do not offer sufficient functional reliability for the intended application.

DE 101 14 205 discloses an apparatus for preventing sleeping in the prone position where the apparatus is not negatively influenced or disturbed in its functionality by movements of the body of a person during sleep, a change in the position of the body of a person during sleep is detected by purely mechanical means or by at least one sensor, and appropriate stimuli are supplied to the body by at least one actuator, so that a predefined target position is assumed during sleep. This apparatus is used to prevent sleeping prone and has a very elaborate construction.

An antisnoring apparatus with means for detecting snoring and a vibrator for halting the snoring process is known from DE 20 2008 009 242. This antisnoring apparatus halts snoring and consists of two units, the first of which is capable of distinguishing snoring from other noises on the basis of characteristics such as frequency, loudness, and cyclic repetition of the individual snoring noises and, after identifying snoring, emitting a time-limited radio signal, while the second unit can be worn on a wrist band like a watch, for example, and, upon receiving the radio signal from the first unit, emits a vibration or contact impulse to the wearer. This known antisnoring apparatus works in practice and is inherently satisfactory but has several drawbacks. For one, it is disadvantageous that the antisnoring apparatus consists of two units, which makes it complicated to handle. Another drawback is that the operator must ensure that the first unit that is supposed to detect the snoring is within reach of the sleeping person so that the snoring process, more particularly the noises produced during this snoring process, can be reliably detected. The danger exists here of the first unit being placed too far from the sleeping person, thus rendering it incapable of reliably detecting the snoring noises. Moreover, it is a laborious task to design the first unit such that it is able to reliably distinguish snoring noises produced during an individual snoring process from other ambient noises (such as breathing sounds or other noises penetrating into or present in the bedroom, for example).

It is therefore the object of the invention to provide an apparatus for detecting an unhealthy position of a recumbent person and with which it is possible to react to the unhealthy position.

It is particularly the object of the invention to provide an antisnoring apparatus that is improved over the antisnoring apparatuses known from the prior art in terms of its operation and effectiveness.

This object is achieved by the features of patent claim 1.

In a first embodiment of the invention, means for detecting a [position of a] recumbent person as well as means for inducing the person to react when an unhealthy position for the recumbent person has been detected by the position-detecting means are present. The means for detecting the position of the recumbent person is embodied in an especially advantageous manner as a position sensor, and the means that induces the person to react is advantageously a vibrator, in which case at least the position sensor and the vibrator are provided in a housing, and the housing is designed to be worn in the chest region of the person. Specifically, this means that, when the position sensor has determined that the person is lying on a side that is adversely affected by arthritis in the shoulder region, the vibrator is activated in order to induce the recumbent person to react, namely to turn to the other side or prone or supine.

Alternatively or in addition to the vibrator that induces the person to react, the means can be a vest that is worn by the person.

The vest has at least one chamber, preferably two chambers that are aligned parallel to one another in the vertical axis of the person, with it being possible for the at least one chamber to be filled, emptied, or maintained in its fill level as a function of the position of the recumbent person. In principle, the position of the recumbent person is detected while recumbent. As long as no position-related unhealthy position is present, it is not necessary to fill the at least one chamber of the vest. Only when an unhealthy position has been detected by the position sensor (for example if the person is lying on a side or supine that is affected by an illness), the at least one chamber of the vest is filled, thereby inciting the person to react, namely to move out of this unhealthy position into another, specifically healthy position. Once this has occurred, the at least one chamber of the vest can be emptied again, or the level of inflation can be maintained or only partially reduced in order to cause the person to remain in this healthy position. The chamber of the vest is advantageously filled with air, in which case a compressor is present that generates the compressed air required for this purpose and supplies it to the at least one chamber. Instead of a compressor, static compressed air generation (using a compressed air bottle, for example) can obviously also be used. Moreover, in addition to the filling or emptying of the at least one chamber of the vest, the vibrator can also be used.

In a development of the invention, the means for detecting the position of the recumbent person is a gyroscope or switch. With a gyroscope as position sensor, it is possible to detect the position of the person continuously (in a stepless manner). This provides a high level of flexibility in detecting the two essential ranges (“healthy” and “unhealthy” position) of the person and taking appropriate measures. Such a gyroscope is an independent building block that can be readily integrated into the apparatus. Instead, the position sensor of another apparatus, such as of a smart phone, can also be used.

Switches are used as especially simple means for detecting the position of the recumbent person. These switches are simple electrical contacts that are provided in a power or signal circuit and close or interrupt the power or signal circuit depending on the position of the recumbent person. This is a measure of whether the person is in a “healthy” or “unhealthy” position. Based on this, measures can be taken either to prevent the person from assuming a prohibited position (that is supposed to be avoided due to an injury or illness, for example), or these measures can be initiated if the person has assumed a prohibited position. These measures are then acoustic, optical, vibratory, or other measures that also include the use of the vest described above that can for example be inflated and deflated.

This switch (two mutually facing contacts or two or more such pairs of contacts, for example) are mounted in a straps or article of clothing that can be worn normally (t-shirt, pajama, or the like) and placed against the body of the person and connected to the power or signal circuit. It is important and essential for the at least one pair of contacts to be mounted in this way in the strap, article of clothing, or the like and put in place by the person such that the two positional ranges of the person to be detected (“healthy” and “unhealthy” position) can be distinguished from one another and appropriate action taken in order to prevent the person from unconsciously moving into a position that is unhealthy, especially when sleeping, because the danger exists in this position of the person injuring himself or because an injury or illness is already present in this position (so that this position is harmful to the person) or the person would begin to snore.

The switch is actuated by the pressure and/or mass of the person's body. It is thus determined simply and automatically that the person has assumed a position that should not be assumed due to the person's state of health and that measures must therefore be taken (activation of the acoustic or vibrator, for example) in order to prevent the person from further maintaining this position. Once these measures have been taken and the person has changed the previously assumed position, the body pressure on the switch is eliminated and the measures that were taken can be discontinued (by deactivating the acoustic or vibratory signal, for example).

The switch can be a button, for example. A button is a switch that is actuated by pressure and then returns to a rest position. A mechanical energy accumulator is used for this purpose, for example. In the case of a switch in the form of a button, it is apparent from the associated effect that it has been actuated. In one embodiment of the apparatus for detecting an unhealthy position of a recumbent person, the at least one switch in the form of a button triggers the vibrations in order to incite the person to change recumbent position. The pressure required to actuate the switch is produced by the person's body mass. The at least one switch can be positioned around the entire girth of the person, for example in the region of his/her spinal column or on the side of his/her body.

With respect to the antisnoring apparatus, according to the invention the means for detecting the snoring process is a position sensor and the position sensor and the vibrator are mounted in a housing, in which case the housing is designed to be worn in a person's chest region.

This design provides the following advantages.

For one, the antisnoring apparatus makes use of the insight that life-threatening apnea (breathing arrest, also referred to here as an “unhealthy” position) and/or snoring noises always occur when a sleeping person lies on his/her back. For this purpose, a position sensor is provided that is suited and designed to detect the position of the sleeping person and distinguish different positions from one another. The vibrator is activated as a function of the detected position of the sleeping person.

If the position sensor detects that the sleeping person is lying face-down or on the side in an uncritical or “healthy” side position, it is not necessary to activate the vibrator, since the apnea of the snoring process does not occur in these positions for known biological reasons. Only when the position sensor detects that the sleeping person is in a critical (“unhealthy”) side or back position can it be assumed due to the biological circumstances that apnea and the snoring process will begin in people who tend to snore or have apnea, for which reason the vibrator is activated in such a case. As a result of this vibration directly on the body of the sleeping person, the person is caused either to breathe and/or stop snoring or to move into a position that is different from the critical (“unhealthy”) side or back position. When this movement has finished, the snoring process in particular is also halted automatically. This is determined by the position sensor that deactivates the vibrator when a movement of the sleeping person from the “unhealthy” position into a different “healthy” position has been detected that was brought about by the previous activation of the vibrator.

In order to improve the manageability of the antisnoring apparatus according to the invention, all of the means for detecting the position of the sleeping person, the vibrator, the power supply, and other means that may be necessary are integrated in one housing. Additional means that may be necessary can include an on/off switch for activating and deactivating the antisnoring apparatus, for example. The arrangement of the required means in one housing ensures that only this one apparatus needs to be worn in the person's chest region. This enables the placement of the apparatus to be simplified substantially, and the apparatus is not a nuisance during sleeping especially when worn in the chest region. Wearing the antisnoring apparatus according to the invention on other areas of the body is not excluded; however, it is especially advantageous for it to be worn in the chest region, since the position of the sleeping person can be detected in an especially reliable manner by the position sensor in this region, and, at the same time, the sleeping person is not bothered by wearing the apparatus in this way.

The use of the vibrator has the special advantage that, for one, the sleeping person receives a clear impulse when lying on his/her back, which is a clear indication that snoring can be expected. Moreover, the vibration offers the advantage that those sleeping with the person are not disturbed. What is more, the position sensor enables the position of the sleeping person to be reliably determined without the possibility of other ambient parameters (such as noises, for example) disrupting the position detection.

In a development of the invention, means are additionally provided that emit an acoustic signal when the person is determined by the position sensor to be lying on his/her back. This makes it possible, for one, for the sleeping person to be alerted not only by the vibration but also by a clear acoustic signal that he/she is on his/her back, which positively means the onset of the snoring process. This is advantageous particularly if the sleeping person is in a slow-wave sleep phase and lying on his/her back, since it is then possible after the supine position is detected to give a clear indication to the sleeping person of this supine position through the vibration as well as through the acoustic signal. Moreover, depending on the sleeping behavior of the person, it can be advantageous if people nearby are alerted to his/her supine position and the imminent snoring process by the acoustic signal.

Moreover, according to the invention the vibrator alone or together with the acoustic-signal emitter can be activated when the person has been determined by the position sensor to be lying on his/her back. Switches are integrated for this purpose into the housing, for example, with which the person can set whether only the vibrator or the vibrator together with the acoustic-signal emitter are to be activated when a supine position is detected during sleep. This switch is mounted in the housing and can be accessed by the operator just like the on/off switch for starting the apparatus. The same applies to the power supply that is also integrated into the housing and can be optionally removed for charging if the power supply is a rechargeable battery. Alternatively or in addition, the power supply can also be a rechargeable battery that is permanently installed in the housing and can be charged from the outside via an appropriate charging port in the housing.

In a development of the invention a strap is or can be fastened to the housing so that the antisnoring apparatus can be placed in a person's chest region. Advantageously, the antisnoring apparatus is an independent apparatus, the necessary means for which (particularly such as the position sensor, vibrator, and power supply) are mounted in the housing, with a strap or the like being fastened to the housing with which the apparatus can be placed in the person's chest region. Such a strap is embodied in an inherently known manner as an elastic band and provided in its end regions with a buckle that can be closed, thus making the apparatus simple to handle when it is being placed in the chest region.

Alternatively, it is conceivable for the antisnoring apparatus to be an independent apparatus to which the strap as described above can be fastened. In such a case, this means that, even though the apparatus forming the antisnoring apparatus is an independent apparatus, it need not be designed solely for this purpose. For example, it is expedient in an advantageous development of the invention for the antisnoring apparatus to be a smart phone.

Such a smart phone usually has a position sensor (also called a gyroscope) and vibrator built in. This means that such a smart phone can be advantageously utilized as an antisnoring apparatus. It only must be ensured that the smart phone is provided with a strap so that it can be placed in the chest region of the sleeping person. As a concrete embodiment, it is conceivable, for example, for the smart phone to be inserted into a receiving pocket, in which case the receiving pocket comprises a strap with which the smart phone can be provided in the person's chest region. In order to be able to use the smart phone as an antisnoring apparatus, all that is needed is to program a suitable app (application) and install it on the smart phone. Using this app, the position sensor of the smart phone can then be used to detect the position of the sleeping person and the vibrator of the smart phone activated when the sleeping person is detected as being in a supine position. The same applies to acoustic-signal emitter that are also integrated into a smart phone.

In another embodiment of an apparatus for detecting an unhealthy position of a recumbent person, a housing holds at least one vibrator, one power supply, and one input/output means. The input/output means is an on/off switch. Moreover, the housing can hold a time-delay switch. The time-delay switch creates an adjustable time interval before the vibrator is or are activated. The time-delay switch is intended to make it possible for the person to change positions without triggering the vibrator. For example, if the person is only supposed to or only wants avoid assuming a supine position, this gives the person the opportunity to change from a right-side position via the supine position (which is unhealthy for them) to a left-side position without triggering vibrations. Only once the time set for the time-delay switch has been exceeded in an unhealthy position are vibrations to be applied to the person, thereby inciting the person to change to a healthy position.

Moreover, an element or controller for adjusting the intensity of the vibrations can be provided in the housing. Since the apparatus for detecting an unhealthy position of a recumbent person is used particularly during the person's sleep phase, the depth of the person's sleep must be taken into consideration here. Moreover, the depth of the partner's sleep must also be considered. In particular, the intensity can selected such that the desired reaction of the person occurs. The desired reaction is to be regarded as a change from an unhealthy position of a recumbent person to a healthy position of a recumbent person. However, waking the person or his/her partner from sleep should be avoided.

In the event that an acoustic signal is provided in order to induce the person to change recumbent positions, the volume of the acoustic signal can be regulated using a volume control.

A strap is used to hold at least one switch for activating the vibrator and to hold the housing. A fastener for displaceably securing the at least one switch and the housing can be integral therewith. The housing and the at least one switch are positioned according to the position of the apparatus for detecting an unhealthy position of a recumbent person on the person's midsection or torso. In order to account for the girth of the person's body depending on the position on the midsection or torso, the strap has a closure. The closure can be a hook-and-loop fastener, snap fastener, buckle, or belt coupler, for example.

The apparatus for detecting an unhealthy position of a recumbent person can be worn by the person like a belt or even in the place of a belt at about the level of his/her navel. The apparatus for detecting an unhealthy position of a recumbent person can also be worn by the person at the level of his/her chest or thorax.

In another embodiment of the apparatus for detecting an unhealthy position of a recumbent person, the at least one switch for detecting the position of the recumbent person is coupled with a transponder. This compact combination of switches and transponder preferably has its own power supply and can be fastened to anywhere on the person's body using adhesive tape, for example. In another embodiment, this compact unit of a switch for detecting the position of the recumbent person, a transponder for transmitting the signal, a power supply, and vibrator in order to incite the person. This compact unit can also be secured to the person's body with the aid of adhesive tapes, for example. The transponder can transmit the signal to an evaluation unit next to the person's bed or to another room. This evaluation unit can, in turn, be coupled with a signal transponder so that other people, such as the nursing staff, can be alerted to the unhealthy position of a recumbent person and measures can be taken to cause the person to assume a healthy recumbent position. Parents can also monitor an unhealthy position of a child who is recumbent. The evaluation or monitoring unit can then be placed in the parents' bedroom. The evaluation or monitoring unit can also be used to monitor the unhealthy position of a recumbent person in a sleep lab.

The vibrator can also be combined with a transponder and can thus be freely secured to the person's body using adhesive tape, for example. The transponder of the vibrator can communicate directly with the transponder of the at least one switch. Alternatively, the transponder of the vibrator can also communicate with the evaluation or monitoring unit. The transponder of the vibrator can thus be controlled and vibrations triggered via the evaluation or monitoring unit. These vibrations are intended to cause the person to change from an unhealthy to a healthy recumbent position.

A time-delay element and a controller for adjusting the intensity of the vibrations can also be integrated into the evaluation or monitoring unit. The intensity of the vibrations is influenced by the radio signal that is transmitted to the transponder of the vibrator. The time-delay element is used to delay the triggering of the vibrations so that the person is able to pass through an unhealthy position while switching from one healthy position to the other without being subjected to vibrations. However, if the person remains in the unhealthy recumbent position, vibrations begin after lapse of the set delay period to cause the person to get out of the unhealthy recumbent position and assume a healthy recumbent position.

A transponder can also be combined with an acoustic signal transponder.

A transponder is a wireless communication apparatus that receives incoming signals and automatically forwards and/or answers them. The transponder can be an active transponder with its own power supply. The use of a passive transponder without its own power supply is also conceivable.

Moreover, the evaluation or monitoring unit can contain a signal transponder for a sound signal, for example. This signal can incite the person directly to change from an unhealthy recumbent position to a healthy recumbent position, provided that the evaluation or monitoring unit is positioned near this person. Alternatively, other people can be alerted by the signal to the unhealthy recumbent position. These people can then take measures in order to incite the person to change from an unhealthy to a healthy recumbent position.

The evaluation or monitoring unit can also be combined with a docking station in order to charge the transponder/switch combination, the transponder/vibrator combination, and/or the transponder/switch/vibrator combination, provided that rechargeable batteries are provided as a power supply. A separate charging apparatus can also be provided, insofar as the mobile elements that can be positioned on the person's body have a rechargeable power supply. A direct data exchange is also conceivable in addition to the data exchange via a wireless connection between the evaluation or monitoring unit and/or docking station. A docking station can also be provided as a separate unit.

In another embodiment of an apparatus for detecting an unhealthy position of a recumbent person, a housing holds at least one vibrator, one power supply, one input/output means, and one transponder. The housing can then be freely positioned on the person's body and secured thereto using adhesive tape, for example. The transponder located in the housing can communicate directly with the switch/transponder combination and/or with the evaluation or monitoring unit.

The apparatus for detecting an unhealthy position of a recumbent person can also be used for people with dementia or for sleepwalkers. A converse mechanism can activate the apparatus for detecting an unhealthy position of a recumbent person as soon as the person sits down in bed, for example. Through a link to other rooms such as staff or relatives' rooms, for example, other people can be alerted to the person's unwanted behavior and take appropriate measures to prevent the person from being hurt. At care facilities and in hospitals, etc., a signal such as an optical or acoustic signal, for example, can be triggered by the apparatus for detecting an unhealthy position of a recumbent person.

According to the invention a means for detecting the position of the recumbent person is provided on the apparatus for detecting an unhealthy position of a recumbent person and means are also present for causing a recumbent person to react when an unhealthy position of the recumbent person has been detected by the position-detecting means, in which case the means for detecting the position of the recumbent person is at least one switch and that the means for inciting the person to a reaction is a vibrator, and that a housing holds at least the vibrator, a power supply, and an input/output means, and that at least one switch and the housing are secured to a strap.

This results in a compact apparatus for detecting an unhealthy position of a recumbent person that is easy for the person to operate. A position sensor such as a gyroscope can be dispensed with in favor of switches. By virtue of his/her simple construction, the switches result in nearly one hundred percent functional reliability.

Moreover, according to the invention the input/output means is an on/off switch. An on/off switch can reliably interrupt the power supply. The vibrator only produces vibrations if the person so desires.

Moreover, according to the invention the at least one switch is a simple electrical contact that is provided in a power or signal circuit and closes or interrupts the power or signal circuit depending on the position of the recumbent person. A very high level reliability is made possible by the simple, robust design.

Moreover, according to the invention the at least one switch is actuated by the person's body pressure. The actuation of the switches by the person's body mass reduces the risk of a malfunction. Only a strong force that is applied to the switches by the body mass of the person triggers the apparatus.

Moreover, according to the invention the power supply is suitable for holding a standard battery or rechargeable battery. Standard batteries ensure functionality even when no charging means are available. All that the person must do is ensure that they have a supply of standard batteries commensurate with the planned absence. If charging means are available, rechargeable batteries are recommended in order to conserve resources.

Furthermore, according to the invention the power supply integrated into the housing can be optionally removed for charging if the power supply is a rechargeable battery. This imparts a high level of flexibility to the power supply, and an external charging apparatus can also be used.

Moreover, according to the invention the power supply is a rechargeable battery that is permanently installed in the housing and can be charged from the outside via an appropriate charging port in the housing. All that the person has to do is establish a connection between the charging apparatus and the apparatus for detecting an unhealthy position of a recumbent person. For example, a USB charger with a USB or micro-USB connector can be used. In this case, a charging apparatus for smart phones or mobile telephones can be used to charge the apparatus for detecting an unhealthy position of a recumbent person. The charging apparatus can then also be operated via a cigarette lighter or on-board electrical outlet of an automobile, for example. A connection to a laptop or PC via a USB connection in order to charge the apparatus for detecting an unhealthy position of a recumbent person is also conceivable.

Moreover, according to the invention the at least one switch is a button. A button has a very simple construction and thus offers a very high level of functional reliability and can be manufactured economically.

Moreover, according to the invention the at least one switch is used to detect a person's side position. Depending on the person's clinical picture, an unhealthy side position can be nearly ruled out by the use of this switch.

Moreover, according to the invention the at least one switch is used to detect a person's supine position. Depending on the person's clinical picture, an unhealthy supine position can be nearly ruled out by the use of this switch.

Moreover, according to the invention the at least one switch is displaceably secured to the strap by fastener. By virtue of the displaceable fastening, the position of the switches can be adapted precisely to the person.

Moreover, according to the invention the fastener is integral with the at least one switch. By virtue of the integral design, the switch is structurally compact and can be manufactured economically.

Moreover, according to the invention the housing is displaceably secured to the strap by fastener. Through the nearly freely selectable position of the housing, the person can choose a suitable position for the housing.

Moreover, according to the invention the fastener is integral with the housing. By virtue of the integral design, the housing is structurally compact and can be manufactured economically.

Moreover, according to the invention the strap has a closure. The closure enables the length of the strap to be adapted precisely to the girth of the person's body. The desired functionality is only ensured through contact of the switch and the vibrator with the person's midsection or torso.

Moreover, according to the invention the at least one switch is connected via an electrically conductive interface to the elements in the housing. All of the electrical elements on the power circuit are conductively interconnected by this interface. Through the flexible design of the conductive interface, the position of the at least one switch and of the housing on the strap in relation to one another can be flexibly selected.

Moreover, according to the invention the closure is a hook-and-loop fastener. A hook-and-loop fastener is simple to manufacture and easy to use by the person.

Moreover, according to the invention the housing has a time-delay switch in addition to the elements. The time-delay switch enables the person to change from one healthy recumbent position to another recumbent position by passing through at least one unhealthy recumbent position therebetween without feeling vibrations on his/her body.

Moreover, according to the invention a controller for regulating the intensity of the vibrations of the vibrator or for regulating the volume of an acoustic signal is provided in the housing. Regulation of the intensity or volume can set the vibrator so that neither the person nor his/her partner is awoken by the vibrations. However, the intensity is to be selected such that the desired effect is achieved.

According to the invention means for detecting the position of the recumbent person are provided on the apparatus for detecting an unhealthy position of a recumbent person and means are also present for causing the person to react when an unhealthy position of a recumbent person has been detected by the position-detecting means, with the means for detecting the position of the recumbent person being at least one switch that is coupled with a transponder.

The use of a transponder ensures that the at least one switch can be secured to nearly any part of the person's body in a simple manner. It is no longer necessary to provide a connection using wires and cables between the individual functional units of the apparatus for detecting an unhealthy position of a recumbent person. Furthermore, the radio signal of the transponder can also be transmitted to other rooms, such as to an evaluation or monitoring unit, for example. Vibrator on the body of the recumbent person coupled with a transponder, for example, can then be controlled from this evaluation or monitoring unit.

The invention is explained in further detail with reference to the enclosed figures.

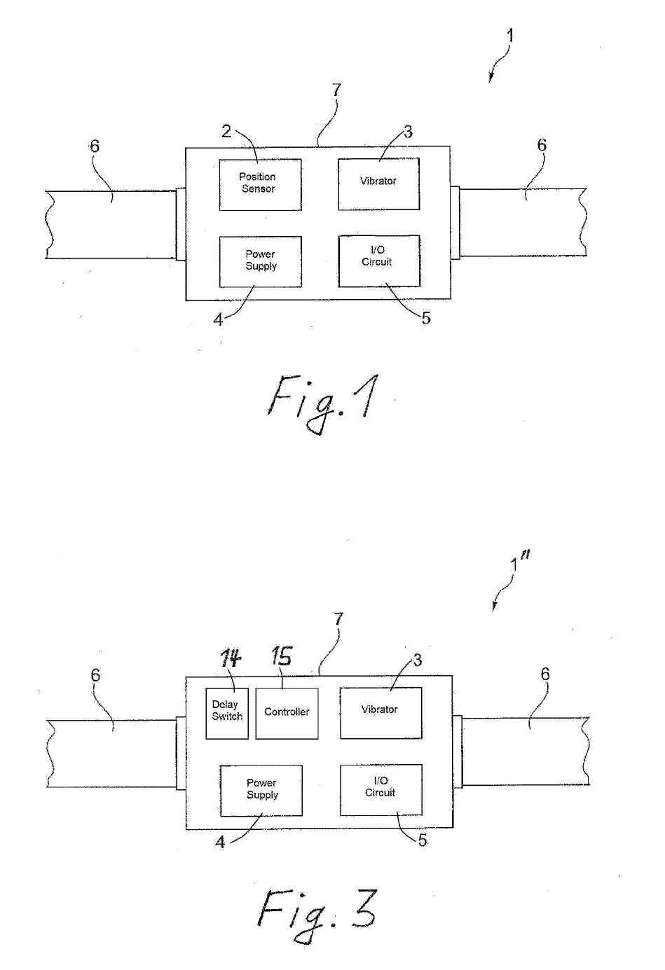

FIG. 1 is a schematic view of an apparatus for detecting an unhealthy position of a recumbent person, particularly an antisnoring apparatus,

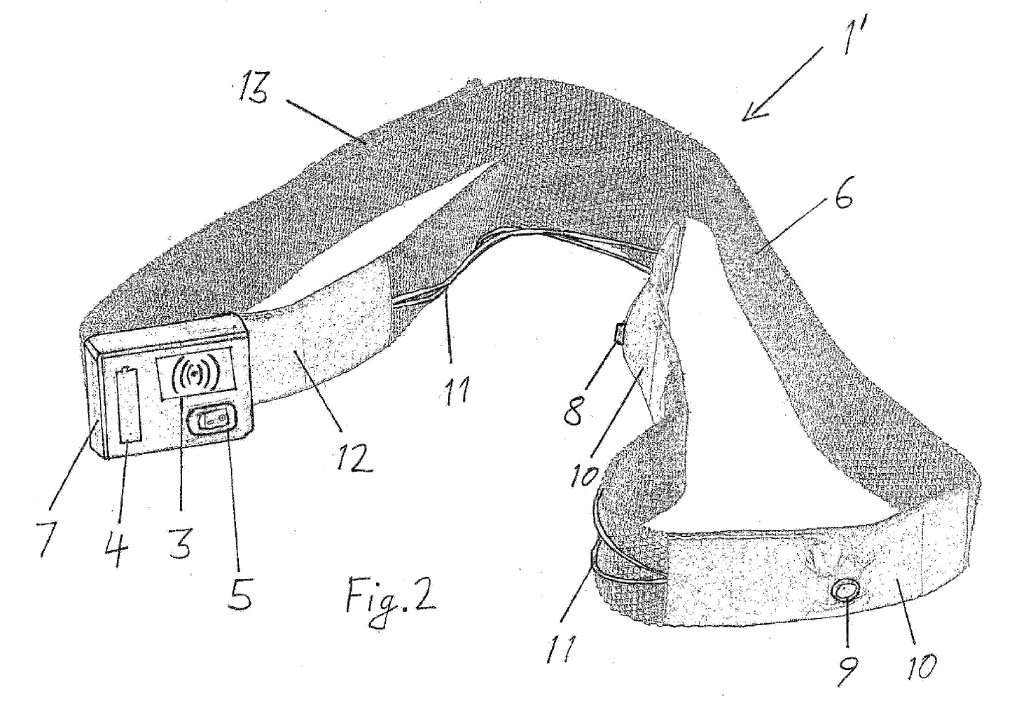

FIG. 2 is another embodiment of an apparatus for detecting an unhealthy position of a recumbent person, and

FIG. 3 is a schematic view of another embodiment of an apparatus for detecting an unhealthy position of a recumbent person.

In the illustrated embodiments, the same references are used for same or functionally analogous elements.

In the following description of the figures, terms such as top, bottom, left, right, front, rear, etc. refer exclusively to the illustration selected in the respective figures and the position of the apparatus and other elements. These terms shall not be understood to be limiting; that is, these references can change as a result of varying positions and/or mirror-inverted design or the like.

In a schematic view, FIG. 1 shows an antisnoring apparatus 1 according to the invention, wherein at least one position sensor 2 and vibrator 3 as well as one power supply 4 must be provided. Optionally, additional input/output means 5 (for example switches, touchscreen for operating an app, and the like) can be provided. Likewise, means can optionally be provided that generate an acoustic signal. The same applies to a switch (not shown) with which a selection can be made by an operator as to whether only the vibrator 3 or, instead, the vibrator 3 and simultaneously also the acoustic-signal emitter are to be actuated when a recumbent position has been detected by the position sensor 2. Finally, a strap 6 is shown schematically that is secured to a housing 7 of the antisnoring apparatus 1 so that the housing 7 can be fastened to the person's chest.

These embodiments of the antisnoring apparatus described above apply in like manner to the apparatus for detecting an unhealthy position of a recumbent person as well.

FIG. 2 shows another embodiment of an apparatus 1′ for detecting an unhealthy position of a recumbent person. The apparatus 1′ has a housing 7. The housing 7 is used to hold a vibrator 3, a power supply 4, and an input/output means 5. The housing 7 is secured to a strap 6 by a fastener 12 that can also be integral with the housing 7. The housing 7 can also be secured to the strap 6 so as to be displaceable. Furthermore, switches 8, 9 are secured to the strap 6 by fastener 10. The switches 8, 9 can also be secured to the strap 6 so as to be displaceable. The switches 8, 9 are connected to the housing 7 via electrically conductive interfaces 11. The apparatus 1′ for detecting an unhealthy position of a recumbent person must have at least one switches 8, 9. Vibrator 3, power supply 4, input/output means 5, and the at least one switches 8, 9 are interconnected in an electrically conductive manner. The input/output means 5 that is an on/off switch in this embodiment, interrupts the power feed to the vibrator 3 and/or the at least one switch 8, 9 when in the off or zero position. If the apparatus 1′ is activated via the input/output means 5 through selection of the “I” position, the power circuit can be closed by actuating at least one switch 8, 9. When the power circuit is closed, the vibrator 3 starts up. The vibrator 3 stops vibrating as soon as either the power circuit is interrupted by the at least one switch 8, 9, the input/output means 5, or the power supply 4. If an exchangeable power supply such as batteries is used, the person must replace the power supply element in the power supply 4 in order to enable the vibrator 3 to be activated again. If the power supply 4 is rechargeable such as for example a rechargeable battery, the person must connect the power supply to a charger. Once a suitable charge state has been reached, the vibrator 3 can be activated again. If the power supply has sufficient electrical energy to power the vibrator and the input/output means 5 is connected and the person activates or deactivates the vibrator 3 using the at least one switch 8, 9. The switches 8, 9 are preferably positioned on the strap 6 such that the person actuates them when on his/her side. This side position may be considered an unhealthy position for a recumbent person in view of the person's discomforts and/or clinical picture. The switch 8 is actuated by the person's body mass, the power circuit is closed, and the vibrator 3 produces vibrations. These vibrations bring the sleeping person to assume a healthy position of a recumbent person. Once the person has assumed this healthy position, the witch 8 interrupts the power circuit, and the vibrator 3 stops vibrating. The switch 9 is preferably positioned on the strap 6 such that the person actuates it as a result of a supine position. This supine position is to be assessed as an unhealthy position of a recumbent person in view of the person's discomforts and/or clinical picture. The switch 9 is actuated by the person's body mass, the power circuit is closed, and the vibrator 3 produces vibrations. These vibrations bring the sleeping person to assume a healthy position of a recumbent person. Once the person has assumed this healthy recumbent position, the switch 9 interrupts the power circuit, and the vibrator 3 stops vibrating.

The apparatus 1′ for detecting an unhealthy position of a recumbent person according to the above-described embodiment comprises at least one vibrator 3, one power supply 4, one input/output means 5, and one switch 8, 9. The elements 3, 4, 5, 8 and/or 9 are preferably mounted on a strap 6. The elements 3, 4, 5 are preferably mounted in a housing 7 that can be in turn mounted on the strap 6. In order to adapt the strap 6 to the girth of the person's body, it has a closure 13. The closure 13 can be a hook-and-loop fastener. An embodiment of the closure 13 as a buckle, push-button, belt couplers, etc., is also conceivable. The housing 7 that preferably contains the elements 3, 4 and 5, and the at least one switch 8, 9 are positioned on the strap 6 according to the girth of the person's body.

FIG. 3 is a schematic view of another embodiment of an apparatus 1″ for detecting an unhealthy position of a recumbent person. The housing 7 can hold a time-delay switch 14 in addition to the elements 3, 4 and 5. The time-delay switch 14 prevents vibrations from being triggered immediately during a desired movement that passes through an unhealthy position of a recumbent person. The person is to be made to change positions only if the person remains in an unhealthy position for an extended period of time.

A controller 15 for varying or regulating the intensity of the vibrations of the vibrator 3 can also be provided in the housing 7. Alternatively, the controller 15 is used to regulate the volume if an acoustic signal transponder is provided. The time-delay switch 14 and the controller 15 are optional; an element 14, 15 can be provided in order to enhance the comfort and functionality of the apparatus 1″ for detecting an unhealthy position of a recumbent person.

The strap 6 can be worn by the person in any position on his/her midsection or torso. The position depends on the requirements of the person's clinical picture and/or his/her preferences.

### LIST OF REFERENCE SYMBOLS

1 apparatus for detecting an unhealthy position of a recumbent person/antisnoring apparatus
1′ apparatus for detecting an unhealthy position of a recumbent person
1″ apparatus for detecting an unhealthy position of a recumbent person
2 position sensor
3 vibrator
4 power supply
5 input/output means
6 strap
7 housing
8 switch
9 switch
10 fastener
11 interface
12 fastener
13 closure
14 time-delay switch
16 controller

1. An apparatus for detecting an unhealthy position of a recumbent person, the apparatus comprising:
means including a switch for detecting the position of the recumbent person;
means including a vibrator for causing the person to react when an unhealthy position of the recumbent person has been detected by the position-detecting means;
a housing that holds at least the vibrator;
a power supply;
an input/output means; and
a strap to which the switch and the housing are secured.

2. The apparatus defined in claim 1, wherein the input/output means is an on/off switch.

3. The apparatus defined in claim 1, wherein the switch is a simple electrical contact provided in a power or signal circuit and closes or interrupts the power or signal circuit depending on the position of the recumbent person.

4. The apparatus defined in claim 1, wherein the switch is actuated by the pressure of the person's body.

5. The apparatus defined in claim 1, wherein the power supply is suitable for holding a standard or rechargeable battery.

6. The apparatus defined in claim 1, wherein the power supply is integrated into the housing, is removable for charging if the power supply, and is a rechargeable battery.

7. The apparatus defined in claim 1, wherein the power supply is a rechargeable battery that is permanently installed in the housing and can be charged from the outside via an appropriate charging port in the housing.

8. The apparatus defined in claim 1, wherein the switch is a button.

9. The apparatus defined in claim 1, wherein the switch is used to detect a person's position on his/her side.

10. The apparatus defined in claim 1, wherein the switch is used to detect a supine position of the person.

11. The apparatus defined in claim 1, further comprising:
a fastener securing the switch is displaceably to the strap.

12. The apparatus defined in claim 11, wherein the fastener is integral with the switch.

13. The apparatus defined in claim 1, further comprising:
a fastener securing the housing displaceably to the strap.

14. The apparatus defined in claim 13, wherein the fastener is integral with the housing.

15. The apparatus defined in claim 1, wherein the strap has a closure.

16. The apparatus defined in claim 1, further comprising:
an electrically conductive interface connecting the position-detecting means, the vibrator, and the input-output circuit to the switch.

17. The apparatus defined in claim 15, wherein the closure is a hook-and-loop fastener.

18. The apparatus defined in claim 1, further comprising:
a time-delay switch in the housing.

19. The apparatus defined in claim 1, further comprising:
a controller in the housing for regulating the intensity of the vibrations of the vibrator or for regulating the volume of an acoustic signal.

20. An apparatus for detecting an unhealthy position of a recumbent person, the apparatus comprising:
means for detecting the position of the recumbent person;
means including a switch for causing the person to react when an unhealthy position of a recumbent person has been detected by the position-detecting means; and
a transponder coupled with the switch.
